(19) United States
(12) Patent Application Publication
(10) Pub. No.: US 2011/0077657 A1
(43) Pub. Date: Mar. 31, 2011

(54) DRILL-AIMING METHOD AND APPARATUS

(75) Inventor: Michael KARASIK, Jerusalem (IL)

(73) Assignee: K.M.B.Y. LTD, Jerusalem (IL)

(21) Appl. No.: 12/887,986

(22) Filed: Sep. 22, 2010

Related U.S. Application Data
(63) Continuation-in-part of application No. PCT/IL2009/000333, filed on Mar. 25, 2009.

(30) Foreign Application Priority Data
Mar. 25, 2008 (IL) 190438

(51) Int. Cl. A61B 17/56

(52) U.S. Cl. 606/97

(57) ABSTRACT

Apparatus and methods are provided for aligning a bone-penetration device with a nail-hole at a distal end of an implantable nail.

A lateral x-ray image of a drill-guide and the distal end of the nail is generated by:
orienting an x-ray source with respect to the drill-guide, using a coupling device; and
generating a lateral x-ray image of the drill-guide and the distal end of the nail, while the x-ray source is oriented with respect to the drill-guide.

It is determined that the drill-guide is aligned with the nail-hole by determining that, in the image:
(a) a line corresponding to a longitudinal axis of the drill-guide, and
(b) a line corresponding to a longitudinal axis of the distal end of the nail are substantially collinear.

In response thereto, the bone-penetration device is aligned using the drill-guide.

### CROSS-REFERENCES TO RELATED APPLICATIONS

The present application is a continuation-in-part of PCT Application PCT/IL09/00333 to Karasik, filed Mar. 25, 2009, which claims priority from Israel Patent Application 190,438 to Karasik, filed Mar. 25, 2008. Both of the aforementioned references are incorporated herein by reference.

### FIELD OF THE INVENTION

The present invention generally relates to orthopedic medical apparatus and methods. Specifically, the present invention relates to apparatus and methods for aligning a bone-penetration device with respect to a nail.

### BACKGROUND OF THE INVENTION

It is a common surgical technique for an elongated metal nail to be implanted along the central portion of a long bone in order to aid the healing of a break in the bone, or shattering of the bone. Subsequent to the insertion of the nail, the nail is typically fastened to the bone using transverse fasteners which extend through the bone and the nail, at both the distal and proximal ends of the nail. The nail typically contains holes at each of its ends for this purpose.

In order to fasten the proximal end of the nail to the bone, typically, a jig with aiming sleeves is fastened to the exposed proximal end of the nail. The jig extends along the bone and the aiming sleeves act as guides for drilling the bone in alignment with the holes in the nail. However, due to bending of the distal end of the nail during insertion of the nail into the bone, the precise position of the holes at the distal end of the nail is unknown. In order to drill holes in the bone through which fasteners can be inserted into the holes at the distal end of the nail, the precise location of the holes at the distal end must be determined.

U.S. Pat. No. 4,418,422 to Richter describes a device for positioning a holder for an aiming sleeve for implanted bone nail fasteners with respect to a Roentgen ray source that includes a support a for supporting the holder. A base plate and a pivotable bracket plate receive the support means and are mountable on the housing of the Roentgen ray source for spacedly positioning the holder from the source. The support is formed as a fork-like element retained in sleeves on the bracket plate by means of locking cams.

U.S. Pat. No. 5,426,687 to Goodall describes a laser targeting device for use during surgical procedures that provides means for the precise co-axial alignment of a narrow laser beam with an x-ray beam. The device has a case which contains a reflecting mirror mounted at 45° on a low mass assembly. The mirror lies directly in front of a radio-opaque cruciform target. The case is adjustably mounted so that the image of the crosshair target may be accurately centered on the target head. A calibration aid is used to pinpoint the emanation point of the x-ray beam from the source cone of the image intensifier. The angle at which the laser beam strikes the mirror may be varied in two planes. When the crosshair image is centered and the laser beam is directed onto the emanation point the two beams are rendered precisely co-axial. Accurate drilling along the laser beam path without further recourse to x-ray screening is described as being more accurate and eliminating direct exposure of the surgeon to irradiation during orthopedic procedures. A series of special drapes is described for the device.

An article entitled “A new technique of distal screw insertion for locked nailing,” by Granhed (Acta Orthop Scand 1998: 69 (3): 320-321) describes the following technique for distal screw insertion for locked nailing. A biplane image intensifier or 2 C-arm is centred over the distal holes of the nail. The lateral intensifier can be positioned 20-45 degrees oblique to the femur. The holes can now be seen, on the AP view, as 2 ovals of the same size on both sides of the nail. If not, the intensifier is rotated or, if the fracture is stable enough, the leg. With an anterior K-wire positioned exactly over the hole, the correct spot for the lateral incision in one plane is decided. By looking at the lateral view, the AP position for the screws can be selected. The incision should be positioned right in the middle of the nail on the screen. Now advance the awl or the drill to the cortex. The drill bit should be perpendicular to the nail and in line with 2 of the opposite ovals, checked with an external K-wire in the AP view. No holes can be seen in the lateral view, but the drill should be positioned in the middle of the nail and in line with it, in the lateral view. Proceed with the drilling and complete the procedure with the screws.

Stryker Corporation (Germany) manufactures the Gamma3 Distal Targeting System, which is described as being designed to facilitate minimally invasive surgery and to reduce the OR time to a minimum with the aid of using the state-of-the art instrumentation and an optimized surgical technique.

The following references may be of interest:

U.S. Pat. No. 4,223,227 to Horwitz

U.S. Pat. No. 4,890,918 to Monford

U.S. Pat. No. 5,031,203 to Trecha

U.S. Pat. No. 5,478,343 to Ritter

U.S. Pat. No. 5,584,838 to Rona

U.S. Pat. No. 6,200,316 to Zwirkoski U.S. Pat. No. 6,877,239 to Leitner

U.S. Pat. No. 7,147,643 to Robioneck

US 2005/0251113 to Kienzle

US 2006/0098851 to Shoham

Japanese Patent Application Publication 2005/237528 to Sawai Toshinao

Japanese Patent Application Publication 2005/237527 to Ota Noboru

“Measuring Human Detection Templates,” by Ahumada et al., NASA Ames Research Center 1997, Research and Technology Report

### SUMMARY OF THE INVENTION

In some embodiments of the present invention, a bone-penetration device (for example, a bone-drill, or a laser) is aligned with a nail-hole at a distal end of an implantable nail. In some embodiments, the nail is an intramedullary nail that is implanted inside a subject's bone, such as the subject's femur. A drill-guide is placed, outside the subject's body, in the vicinity of the hole at the distal end of the nail. At least one lateral x-ray image of the drill-guide and the distal end of the nail is generated. It is determined that the drill-guide is aligned with the hole by determining that, in the lateral x-ray image:
(a) a line corresponding to a longitudinal axis of the drill-guide and
(b) a line corresponding to a longitudinal axis of the distal end of the nail are substantially collinear, i.e., the two lines substantially lie along a common line.

The bone-penetration device is aligned with the hole, using the drill-guide. For example, in response to determining that the drill-guide is aligned with the hole, a drill, or another bone penetration device (such as a laser), may be inserted into the drill-guide. Or, the drill-guide may be aligned with the hole, the bone-penetration device (e.g., the drill) having been inserted into the drill-guide.

Typically, before generating the lateral x-ray, the x-ray source, from which the x-rays are generated, is oriented with respect to the drill-guide. The x-ray source is oriented using a coupling device that couples the drill-guide to the x-ray source. For example, the x-ray source may be oriented using an optical-coupling device that optically couples the drill-guide to the x-ray source.

In some embodiments, the drill-guide is aligned with the hole at the distal end of the nail by generating a plurality of lateral x-ray images of the drill-guide and the distal end of the nail. Based upon the relative positions of projections of the drill-guide and the distal end of the nail in the first x-ray image, the position of the drill-guide is adjusted. Subsequently, the position of the x-ray source is adjusted so that the x-ray source is oriented with respect to the drill-guide in a similar orientation to the orientation of the x-ray source with respect to the drill-guide, during the generation of the first x-ray image. A second lateral x-ray image of the drill-guide and the distal end of the nail is generated, while the drill-guide and the x-ray source are in their new positions. In some embodiments, it is determined that the drill-guide is aligned with the hole by determining that in the second lateral x-ray image:
(a) the line corresponding to a longitudinal axis of the drill-guide and
(b) the line corresponding to a longitudinal axis of the distal end of the nail are substantially collinear.

Alternatively, in response to detecting that in the second lateral x-ray image lines (a) and (b) are not substantially collinear, the positions of the drill-guide and the x-ray source are further adjusted. The positions of the drill-guide and the x-ray source having been further adjusted, further lateral x-ray images are generated, until it is determined that, in a lateral x-ray image, lines (a) and (b) are substantially collinear.

Typically, in order to determine that the drill-guide and the hole are aligned, in addition to using the lateral x-ray image, at least one frontal x-ray image of the drill-guide and the distal end of the nail is used. Typically, the lateral x-ray image is used to determine that the drill-guide and the hole are aligned in a first plane, and the frontal x-ray image is used to determine that the drill-guide and hole are aligned in a second plane. In some embodiments, the drill-guide is first aligned with the hole in the second plane using the frontal x-ray images, and, subsequently, the drill-guide is aligned with the hole in the first plane, using the lateral x-ray images. Alternatively, the drill-guide is first aligned with the hole in the first plane using the lateral x-ray images, and subsequently, the drill-guide is aligned with the hole in the second plane, using the frontal x-ray images.

In some embodiments, before generating the frontal x-ray image, the x-ray source is oriented with respect to the drill-guide, using a coupling device that couples the x-ray machine to the drill-guide. For example, an optical-coupling device that optically couples the x-ray machine to the drill-guide may be used. The frontal x-ray image is generated using the x-ray source, after the x-ray source has been oriented with respect to the drill-guide. It is subsequently determined that the drill-guide is aligned with the hole by determining that, in the frontal x-ray image, projections of the drill-guide and the hole are substantially collinear. In some embodiments, a plurality of frontal x-ray images of the drill-guide and the distal end of the nail are generated. The positions of the drill-guide and the x-ray source are adjusted in response to the relative positions of projections of the drill-guide and the distal end of the nail in the x-ray images, until the projections of the drill-guide and the hole appear as being substantially collinear in an x-ray image.

Although embodiments are described with respect to a drill-guide for guiding a mechanical drill, the scope of the present invention includes using guiding apparatus that is suitable for a different bone-penetration device, such as a laser, which can “drill” into bone, mutatis mutandis. Thus, in embodiments in which a laser is used, the term “drill-guide” refers to a guide that holds the laser device in place.

It is noted that embodiments of the present invention are described with respect to lateral x-ray images. In this context, the scope of the present invention includes using lateral x-ray images that are taken from any lateral position with respect to the subject, including from an oblique angle to the portion of the subject's body that is imaged. For example, the scope of the present invention includes acquiring a lateral x-ray image of the subject's femur by positioning an x-ray source adjacent to the subject's shoulder, and positioning an x-ray detector between the subject's legs.

It is noted that embodiments of the present invention are described with respect to holes in an implantable nail, the longitudinal axes of the holes being in a generally lateral direction. However the scope of the present invention includes applying the techniques described herein to an implantable nail that defines holes, the longitudinal axes of the holes being in a generally anterior-posterior direction, mutatis mutandis. For example, it may be determined that a drill-guide is aligned with such holes in a nail, by determining that, in a lateral x-ray image, projections of the drill-guide and the hole are substantially collinear. It may further be determined that a drill-guide is aligned with such holes in a nail, by determining that, in a frontal x-ray image:
(a) the line corresponding to a longitudinal axis of the drill-guide and
(b) the line corresponding to a longitudinal axis of the distal end of the nail are substantially collinear.

In some embodiments of the present invention, a method is provided for precisely setting a drill for drilling a hole in a bone in which a nail has been inserted, in order to obtain a hole coaxial with a hole in the nail, for subsequent fixation of the nail in the bone by means of a screw. In some embodiments, devices for realizing this method are provided. In the context of the present application, a drill should be interpreted as any instrument that may be used for making holes in bones, e.g., a cylindrical-shaped bone-penetration device.

In some embodiments, a drill-guide is provided for guiding the bone-penetration device during the creation of the hole in the bone. In some embodiments, the drill-guide position is corrected based upon frontal and lateral x-ray images of the drill-guide and the distal end of the nail. The lateral x-ray images are typically acquired at an angle such that the projection of the drill-guide looks like an elongated figure having parallel edges. The appearance of the projection of the drill-guide in this manner allows one to determine in what manner the drill-guide should be rotated and/or translated. The drill-guide is rotated and translated such that that the hole in the bone that is formed by a bone-penetration device that is guided by the drill-guide, is coaxial with the hole in the nail. Typically, the drill-guide is rotated and translated such that:
(a) a line corresponding to a longitudinal axis of the drill-guide and
(b) a line corresponding to a longitudinal axis of the distal end of the nail are substantially collinear, in the lateral x-ray image.

Before acquiring each x-ray image, the x-ray machine is oriented with respect to a drill-guiding device of the drill-guide, using a coupling device, such as an optical-coupling device. The x-ray machine is oriented such that an x-ray generated by the machine is perpendicular to definite planes of the drill-guiding device, the positions of the definite planes being fixed with respect to the drill-guide. The orientation of the x-ray machine with respect to the drill-guide is typically such that distortions that are inherent to x-ray images are reduced.

The correction of the drill-guide position, based on the x-ray images, is typically performed by measuring angles and distances between the axes of the projections of the drill-guide and the distal end of the nail in frontal and lateral x-ray images. For example, in response to a lateral x-ray image in which (a) the line corresponding to a longitudinal axis of a projection of the drill-guide and (b) the line corresponding to a longitudinal axis of the distal end of the nail, are not collinear, the following procedure is performed. The angle between lines (a) and (b) measured on a lateral x-ray image is typically corrected by a correction function, e.g., a correction coefficient that is determined empirically or geometrically for a given apparatus. A correction function is typically utilized due to the fact that the lateral x-ray image is acquired from such an angle with respect to the longitudinal axis of the drill-guide, that the projection of the drill-guide looks like an elongated figure. After rotating the drill-guide through a correction angle, the distance between the axes of the projections of the drill-guide and hole in the nail is measured, and the drill-guide is translated such that that the axes of the projections of the drill-guide and the hole in the nail coincide. In some embodiments, further frontal and/or lateral x-ray images are generated after each displacement of the drill-guide, and the procedure of the correction of the drill-guide position is repeated. For some applications, after several iterations, essentially complete coincidence of the axes of the projections of the drill-guide and the hole in the nail in frontal and lateral x-ray images is achieved.

In some embodiments, an aiming-device for drill setting, which realizes the method described hereinabove, includes an x-ray machine, a drill-guiding facility (also referred to herein as drill-guide apparatus) and a device for optically-coupling the x-ray machine with the drill-guiding facility. For some applications, the drill-guiding facility is fastened to the nail handle. For some applications, the drill-guiding facility includes:

at least one drill-guide holder with drill-guide holes for the drill-guide, wherein the number and spacing of the holes correspond to the number and spacing of the holes in the nail;

a rough-aiming unit for approximately positioning the drill-guide holder with the drill-guide; and

a precise-aiming unit for precisely positioning the drill-guide holder with the drill-guide.

In some embodiments, the precise aiming unit allows the translation of said drill-guide in at least two perpendicular planes and its rotation about at least two axes perpendicular to said planes.

For some applications, the optical-coupling device comprises:

a light source that emits a narrow beam of light, the source being fastened to the receiver of the x-ray machine such that the emitted beam is parallel to the central x-ray beam emitted by the x-ray machine;

a semi-transparent mirror or a mirror with a hole in the center fastened on the receiver of the x-ray machine so that the light-source beam passes through it, the mirror plane being perpendicular to the light beam; and

two mirrors fastened to the drill-aiming device such that they are displaced in the same manner as the drill-guide, when the drill-guide is displaced. The mirrors are arranged at such angles that the x-ray machine is aimed using one of the mirrors before acquiring a frontal x-ray image and by the other mirror before acquiring a lateral x-ray image. In some embodiments, the x-ray machine is aimed by making the incident and reflected light beams of the light source coincide using coincidence marks made on each mirror.

There is therefore provided, in accordance with some applications of the present invention, a method for aligning a bone-penetration device with a nail-hole at a distal end of an implantable nail, the method including:

generating at least one lateral x-ray image of a drill-guide and the distal end of the nail by:
orienting an x-ray source of an x-ray machine with respect to the drill-guide, using a coupling device that couples the x-ray machine to the drill-guide; and
generating at least one lateral x-ray image of the drill-guide and the distal end of the nail, using the x-ray source, while the x-ray source is oriented with respect to the drill-guide;

determining that the drill-guide is aligned with the nail-hole by determining that, in the lateral x-ray image:
(a) a line corresponding to a longitudinal axis of the drill-guide and
(b) a line corresponding to a longitudinal axis of the distal end of the nail are substantially collinear; and

aligning the bone-penetration device using the drill-guide, in response to determining that the drill-guide is aligned with the nail-hole.

For some applications, determining that the drill-guide is aligned with the nail-hole further includes determining that, in the x-ray image, a projection of a radiopaque indicating element that is coupled to the drill-guide appears in a predefined geometrical relationship with respect to a projection of the distal end of the nail.

For some applications, orienting the x-ray source of the x-ray machine with respect to the drill-guide includes orienting the x-ray source such that, when the drill guide is aligned with the nail hole, a central portion of the x-ray beam coincides with a point at which the line corresponding to the longitudinal axis of the drill guide coincides with the nail hole.

For some applications, orienting the x-ray source with respect to the drill-guide includes optically coupling the drill-guide to the x-ray machine using an optical-coupling device that optically couples the x-ray machine to the drill-guide.

For some applications, using the optical-coupling device includes detecting that an incident light beam emitted by a light source of the optical-coupling device coincides with a reflection of the incident light beam that is reflected from a mirror of the optical-coupling device.

For some applications, using the optical-coupling device includes determining that a mirror of the optical-coupling device that is physically coupled to the drill-guide is aligned with a mirror of the optical-coupling device that is physically coupled to the x-ray machine.

For some applications, using the optical-coupling device includes determining that a mirror of the optical-coupling device that is physically coupled to the drill-guide is aligned with a non-mirrored surface of the optical-coupling device that is physically coupled to the x-ray machine.

For some applications, using the optical-coupling device includes determining that a non-mirrored surface of the optical-coupling device that is physically coupled to the drill-guide is aligned with a mirror of the optical-coupling device that is physically coupled to the x-ray machine.

For some applications, in generating the at least one lateral x-ray image and determining that the drill-guide is aligned with the nail-hole, the method includes:

generating a first lateral x-ray image, while the drill-guide is in a first drill-guide position, and while the x-ray source is in a first x-ray source position;

using the first lateral x-ray image, determining if the drill-guide is not aligned with the nail-hole;

in response to determining that the drill-guide is not aligned with the nail-hole, moving the drill-guide from the first drill-guide position to a second drill-guide position, in order to align the drill-guide with the nail-hole;

using the coupling device, moving the x-ray source from the first x-ray source position to a second x-ray source position, such that the x-ray source is oriented with respect to the drill-guide, while the drill-guide is in the second drill-guide position;

while the drill-guide is in the second drill-guide position, and the x-ray source is in the second x-ray source position, generating a second lateral x-ray image of the drill-guide and the distal end of the nail, using the x-ray source; and

determining that the drill-guide is aligned with the nail-hole using the second x-ray image.

For some applications, moving the drill-guide from the first drill-guide position to the second drill-guide position includes, in response to the first x-ray image, moving the drill-guide in accordance with drill-guide motion data disposed in a table of drill-guide motions.

For some applications, moving the drill-guide from the first drill-guide position to the second drill-guide position includes, in response to the first x-ray image, determining a manner in which to move the drill-guide using a computer.

For some applications, determining the manner in which to move the drill-guide using the computer includes providing the first x-ray image as input to the computer.

For some applications,

the drill-guide includes a drill-guide that is insertable through at least two drill-guide holes of at least one drill-guide holder, longitudinal axes of the drill-guide holes being parallel to each other and defining a drill-guide plane, and

orienting the drill-guide with respect to the x-ray source includes placing the drill-guide such that a central ray of an x-ray beam of the x-ray source is in an orientation selected from the group consisting of: parallel to the drill-guide plane, and in the drill-guide plane.

For some applications, the nail includes a nail that defines at least first and second nail-holes at the distal end thereof, and determining that the drill-guide is aligned with the nail-hole includes determining that a second drill-guide hole of the at least two drill-guide holes is aligned with the second nail-hole, by determining that a first drill-guide hole of the at least two drill-guide holes is aligned with the first nail-hole.

For some applications, the method further includes:

orienting an x-ray source of an x-ray machine with respect to the drill-guide, using a coupling device that couples the x-ray machine to the drill-guide; and

generating at least one frontal x-ray image of the drill-guide and the distal end of the nail, using the x-ray source, while the x-ray source is oriented with respect to the drill-guide,

and determining that the drill-guide is aligned with the nail-hole further includes determining that, in the frontal x-ray image, the drill-guide and the nail-hole are substantially collinear.

For some applications,

the drill-guide includes a drill-guide that is insertable through at least two drill-guide holes of at least one drill-guide holder, longitudinal axes of the drill-guide holes being parallel to each other and defining a drill-guide plane, and

orienting the drill-guide with respect to the x-ray source includes orienting the drill-guides such that the drill-guide plane is a plane that is perpendicular to a central ray of an x-ray beam of the x-ray source.

There is further provided, in accordance with some applications of the present invention, apparatus for aiming a bone-penetration device and for use with (a) an x-ray machine including an x-ray source and an x-ray receiver, and (b) a nail that is implanted in a bone of a subject, the nail defining at least one nail-hole at a distal end thereof, the apparatus including:

a drill-guide configured to align the bone-penetration device with the nail-hole; and

an optical coupling device that optically couples the drill-guide to the x-ray machine and is configured to facilitate orientation of the drill-guide with respect to the x-ray source, during acquisition of a lateral x-ray image, such that it may be determined that the drill-guide is aligned with the nail-hole by determining that, in the lateral x-ray image:
(a) a line corresponding to a longitudinal axis of the drill-guide and
(b) a line corresponding to a longitudinal axis of the distal end of the nail are substantially collinear.

For some applications, the apparatus further includes at least one radiopaque indicating element coupled to the drill-guide and configured to facilitate verification of alignment of the drill-guide with the nail-hole by a projection of the indicating element appearing in an x-ray image of the nail in a predefined geometrical relationship with respect to the nail when the drill-guide is properly aligned with the nail-hole.

For some applications, the coupling device includes an optical-coupling device including a first portion physically coupled to the drill-guide and a second portion physically couplable to the x-ray machine, and configured to couple the drill-guide to the x-ray machine by optically coupling the x-ray machine to the drill-guide.

For some applications, the first portion of the optical-coupling device includes a drill-guide mirror that is physically coupled to the drill-guide.

For some applications, the second portion of the optical-coupling device includes a light source, and the optical-coupling device is configured to facilitate the orientation of the drill-guide with respect to the x-ray source, by an incident light beam that is emitted from the light source coinciding with a reflected beam of the incident beam that is reflected from the mirror.

For some applications, the second portion of the optical-coupling device includes a non-mirrored surface that is physically couplable to the x-ray machine.

For some applications, the drill-guide mirror includes:

a first drill-guide mirror that is coupled to the drill-guide and configured to facilitate orientation of the x-ray source with respect to the drill-guide, during acquisition of a frontal x-ray that is taken by the x-ray machine, and

a second drill-guide mirror that is coupled to the drill-guide and configured to facilitate the orientation of the x-ray source with respect to the drill-guide, during acquisition the lateral x-ray that is taken by the x-ray machine.

For some applications, the second portion of the optical-coupling device includes an x-ray machine mirror.

For some applications, the drill-guide mirror and the x-ray machine mirror have markings on respective faces thereof, the markings facilitating rotational alignment of the drill-guide mirror and the x-ray machine mirror.

For some applications, the second portion of the optical-coupling device includes an x-ray machine mirror that is physically couplable to the x-ray machine.

For some applications, the first portion of the optical-coupling device includes a non-mirrored surface that is physically coupled to the drill-guide.

For some applications, the apparatus further includes an x-ray machine mirror housing, operative to physically couple the x-ray machine mirror to the x-ray machine such that a face of the x-ray machine mirror is contained in a plane that is perpendicular to the central ray of the x-ray beam.

For some applications, the first portion of the optical-coupling device includes at least one light source that is physically coupled to the drill-guide, and the optical-coupling device is configured to facilitate the orientation of the x-ray source with respect to the drill-guide, by an incident light beam that is emitted from the light source coinciding with a reflected beam of the incident beam that is reflected from the mirror.

For some applications,

the apparatus further includes at least one drill-guide holder shaped to define at least first and second drill-guide holes, the drill-guide being configured to be inserted through the drill-guide holes, longitudinal axes of the first and second drill-guide holes being parallel to each other and defining a drill-guide plane,

the coupling device being configured to facilitate orientation of the x-ray source with respect to the drill-guide, during acquisition of the lateral x-ray image, such that a central ray of an x-ray beam of the x-ray source is in an orientation selected from the group consisting of: parallel to the drill-guide plane, and in the drill-guide plane.

For some applications, the nail includes a nail that defines at least first and second nail-holes at the distal end thereof, and a distance between the first drill-guide hole and the second drill-guide hole is the same as a distance between the first nail-hole and the second nail-hole.

For some applications, the coupling device is further configured to facilitate orientation of the x-ray source with respect to the drill-guide, during acquisition of a frontal x-ray image, such that it may be determined that the drill-guide is aligned with the nail-hole by determining that, in the frontal x-ray image, the drill-guide and the nail-hole are substantially collinear.

For some applications,

the apparatus further includes at least one drill-guide holder shaped to define at least first and second drill-guide holes, the drill-guide being configured to be inserted through the drill-guide holes, longitudinal axes of the first and second drill-guide holes being parallel to each other and defining a drill-guide plane,

the coupling device being configured to facilitate orientation of the x-ray source with respect to the drill-guide, during acquisition of the frontal x-ray image, such that the drill-guide plane is a plane that is perpendicular to a central ray of an x-ray beam of the x-ray source.

There is additionally provided, in accordance with some applications of the present invention, a method for aligning a bone-penetration device with a nail-hole at a distal end of an implantable nail, the method including:

orienting an x-ray source of an x-ray machine with respect to a drill-guide, using a optical coupling device that optically couples the x-ray machine to the drill-guide;

generating a first frontal x-ray image of the drill-guide and the distal end of the nail, using the x-ray source, while the x-ray source is oriented with respect to the drill-guide, while the drill-guide is in a first drill-guide position, and while the x-ray source is in a first x-ray source position;

using the first frontal x-ray image, determining if the drill-guide is not aligned with the nail-hole;

in response to determining that the drill-guide is not aligned with the nail-hole, moving the drill-guide from the first drill-guide position to a second drill-guide position, in order to align the drill-guide with the nail-hole;

using the coupling device, moving the x-ray source from the first x-ray source position to a second x-ray source position, such that the x-ray source is oriented with respect to the drill-guide, while the drill-guide is in the second drill-guide position;

while the drill-guide is in the second drill-guide position, and the x-ray source is in the second x-ray source position, generating a second frontal x-ray image of the drill-guide and the distal end of the nail, using the x-ray source; determining that the drill-guide is aligned with the nail-hole by determining that, in the second frontal x-ray image, the drill-guide and the nail-hole are substantially collinear; and

aligning the bone-penetration device using the drill-guide, in response to determining that the drill-guide is aligned with the nail-hole.

For some applications, the x-ray machine is not physically coupled to the drill-guide.

The present invention will be more fully understood from the following detailed description of embodiments thereof, taken together with the drawings, in which:

### BRIEF DESCRIPTION OF THE DRAWINGS

FIGS. 1A-C are schematic illustrations of a thought experiment, conducted in accordance with an embodiment of the present invention;

FIG. 2 is a schematic illustration of an implanted nail, drill-guide apparatus, and an x-ray machine, in accordance with an embodiment of the present invention;

FIG. 3 is an enlarged schematic illustration of the drill-guide apparatus, in accordance with an embodiment of the present invention;

FIG. 4 is a schematic illustration of the drill-guide apparatus oriented with respect to an x-ray source of the x-ray machine, during a lateral x-ray, in accordance with an embodiment of the present invention;

FIGS. 5A-B are schematic illustrations of the view from the x-ray source, which would produce lateral x-ray images of projections of the drill-guide and the distal end of the nail when the drill-guide is respectively misaligned and aligned with the hole at the distal end of the nail, in accordance with an embodiment of the present invention;

FIG. 6 is a schematic illustration of the drill-guide apparatus oriented with respect to the x-ray source during a frontal x-ray, in accordance with an embodiment of the present invention;

FIGS. 7A-B are schematic illustrations of the view from the x-ray source, which would produce frontal x-ray images of projections of the drill-guide and the distal end of the nail, when the drill-guide is respectively misaligned and aligned with a hole at the distal end of the nail, in accordance with an embodiment of the present invention;

FIG. 8 is a schematic illustration of the drill-guide apparatus, including radiopaque indicating elements, in accordance with an embodiment of the present invention; and

FIGS. 9 and 10 are schematic illustrations of the view from the x-ray source, which would produce, respectively, frontal and lateral x-ray images of projections of the drill-guide, the distal end of the nail and the radiopaque indicating elements, in accordance with an embodiment of the present invention.

### DETAILED DESCRIPTION OF EMBODIMENTS

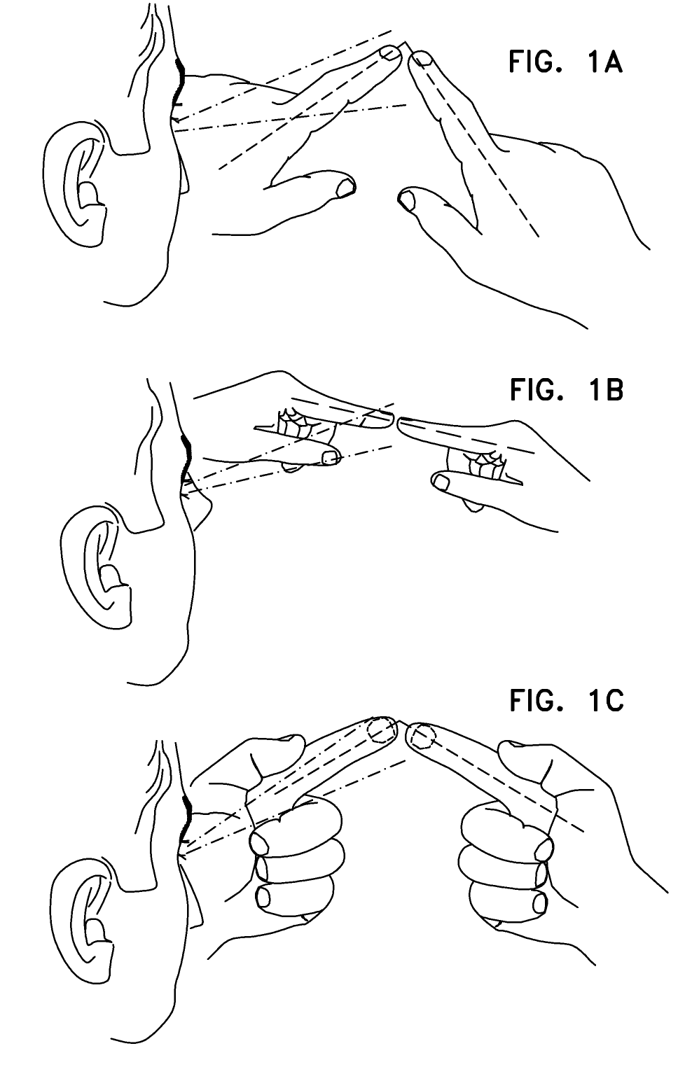

Reference is now made to FIGS. 1A-C, which are schematic illustrations of a thought experiment, conducted in accordance with an embodiment of the present invention. If one places one's index fingers, as shown in FIG. 1A, such that the two index fingers are at a right-angle (for example) to each other, then both fingers are disposed in the plane in which the right-angle is formed, and the following may be observed. (The described effect is not limited to when the index fingers are placed at a right-angle to each other, rather the effect occurs at whichever angle the index fingers are placed with respect to each other.) When the eye views the two fingers from within the plane in which the angle is formed, i.e., the plane that is common to both fingers, then the fingers appear substantially collinear, i.e., the fingers form a straight line with each other, as shown in FIG. 1B. When the two fingers are viewed from below the plane in which the right-angle is formed, the two fingers appear as a V-shape, as shown in FIG. 1C. Similarly, when the two fingers are viewed from above the plane in which the right-angle is formed, the two fingers appear as a V-shape, as shown in FIG. 1A.

Thus, if one wished to determine whether the two index fingers were located in a common plane, a simple test would be to observe the fingers from a plane in which one of the fingers is located. If the two fingers are in a common plane, then the eye of the observer views both fingers within the common plane. Therefore, the fingers will appear as forming a straight line with each other, as shown in FIG. 1B. If the two fingers are in parallel planes to each other, the parallel planes being at different heights, then the two fingers will appear as being parallel to each other, but not collinear. If the two fingers are in planes that are not parallel to each other, then the two fingers will appear as not being parallel to each other.

In some embodiments of the present invention, apparatus and methods are provided for aligning a drill-guide with a hole in the distal end of a nail that is implanted within a subject's bone. A frontal x-ray is generated to determine that, from a frontal view, the longitudinal axis of the drill-guide is aligned with the longitudinal axis of the hole. However, for the drill-guide to be fully aligned with the hole, the longitudinal axis of the drill-guide must also be disposed in the same plane as the longitudinal axis of the hole.

In order to determine whether the drill-guide and the nail define a common plane, an x-ray source generates a lateral x-ray image of the drill-guide and the distal end of the nail, while the x-ray source is disposed in a plane in which the drill-guide is disposed. It is determined that the drill-guide and the nail define a common plane if, in the x-ray image,
(a) the line corresponding to a longitudinal axis of the drill-guide and
(b) the line corresponding to a longitudinal axis of the distal end of the nail are substantially collinear.

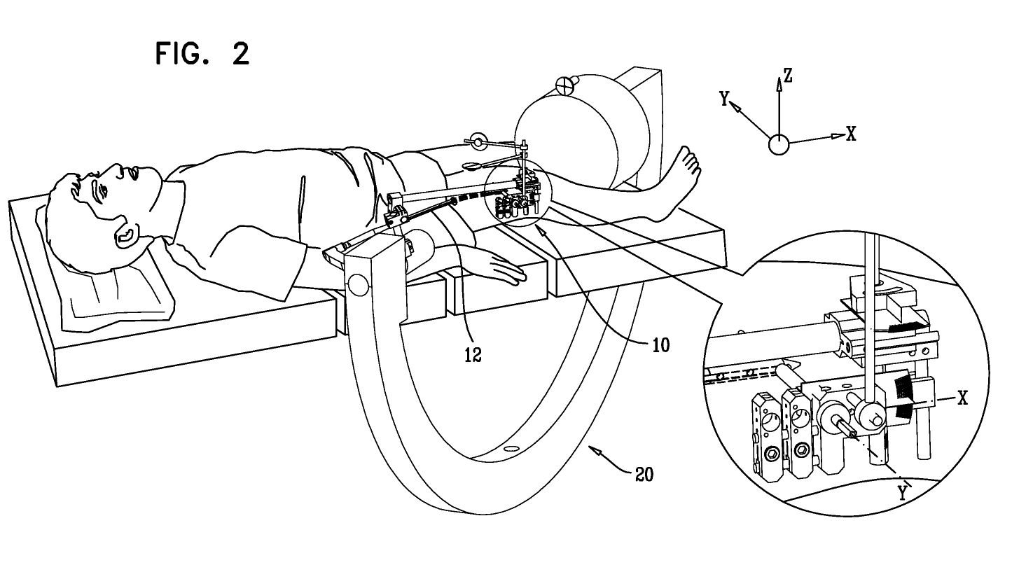

Reference is now made to FIG. 2, which is a schematic illustration of an implanted nail 12, drill-guide apparatus 10, and an x-ray machine 20, in accordance with an embodiment of the present invention. Typically, the x-ray machine generates x-ray images of the drill-guide and the distal end of the nail, after the nail has been implanted inside the subject's bone. It is determined that the drill-guide is aligned with a hole at the distal end of the nail by means of the generated x-ray images.

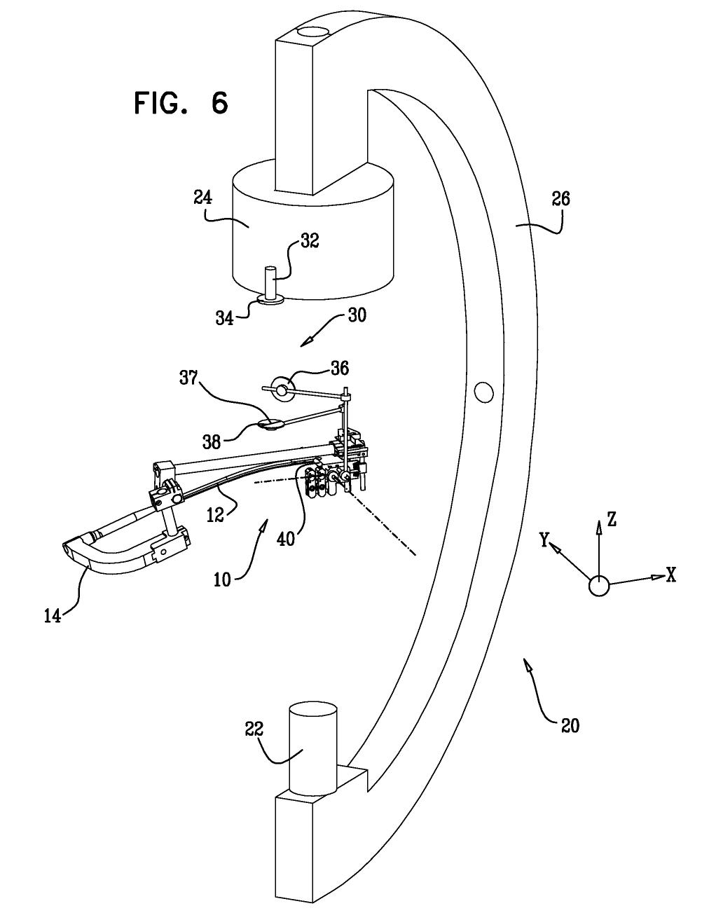

Typically, the drill-guide is aligned in the x-y plane, with the hole in the nail, by means of a frontal x-ray (shown in FIG. 6, for example), the x and y axes being as defined hereinbelow. The drill-guide is aligned in the y-z plane, with the hole, by means of a lateral x-ray, as shown in FIG. 2, the y and z axes being as defined hereinbelow.

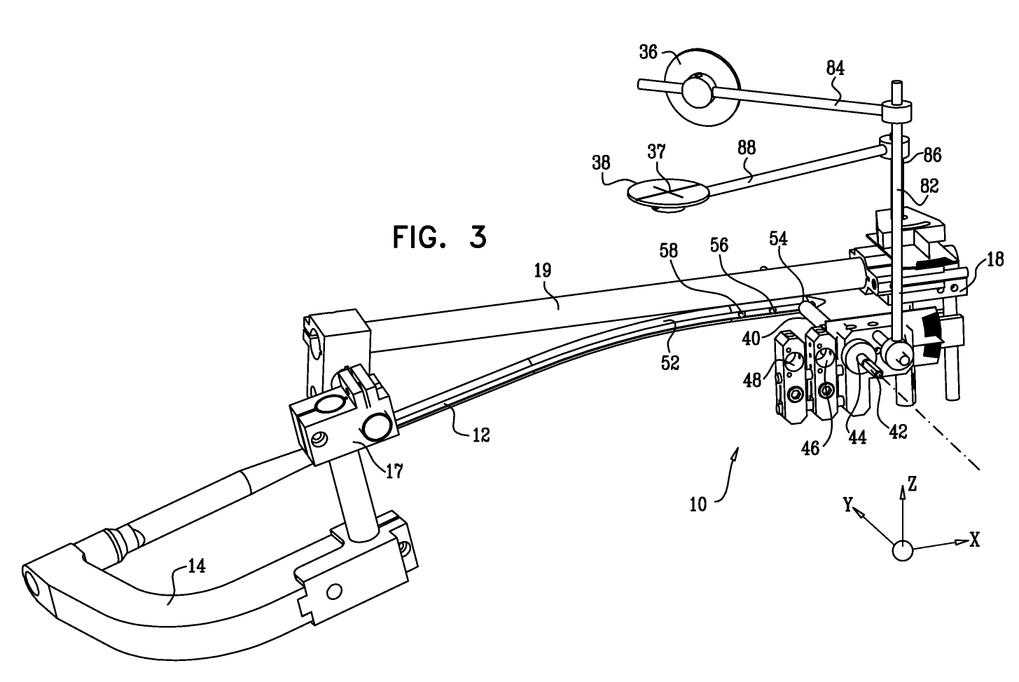

FIG. 3 is an enlarged schematic illustration of drill-guide apparatus 10, in accordance with an embodiment of the present invention. Drill-guide apparatus 10 typically includes a handle 14 coupled to nail 12. Drill-guide apparatus 10 comprises at least one drill-guide holder shaped to provide drill-guide holes 44, 46, and 48. The distances between the centers of drill-guide holes 44, 46 and 48 are equal to the distances between nail-holes 54, 56 and 58 in distal end 52 of nail 12. (Distal end 52 of nail 12 is typically inflexible and maintains a straight configuration even as it is implanted inside the subject's bone.) A drill-guide 40 is placed in drill-guide hole 44, and a drill or simulated drill 42 is placed inside drill-guide 40. (In some embodiments, the drill is placed inside drill-guide 40 only after the drill-guide has been aligned with nail-hole 54, as described hereinbelow.) In the context of the present application, the drill-guide plane is defined as the plane of the axes of at least two of drill-guide holes 44, 46 and 48. In the context of the present application, the y-axis is defined by the longitudinal axis of the drill-guide, the x-axis is defined as the axis that is perpendicular to the y-axis in the drill-guide plane, and the z-axis is perpendicular to the x-axis and the y-axis.

Drill-guide apparatus 10 typically includes a rough-aiming unit 17 of drill-guide 40 and a precise-aiming unit 18. The rough-aiming unit orients a pole 19, with respect to the nail, the drill-guide being coupled to the nail via pole 19, rough-aiming unit 17, and handle 14, as shown in FIG. 3. By orienting pole 19, the rough-aiming unit places drill-guide 40 in the vicinity of distal end 52 of nail. Precise-aiming unit precisely aligns the drill-guide with nail-hole 54 at the distal end of the nail.

Precise-aiming unit 18 typically allows the drill-guide to be rotated around two axes. In addition, the precise-aiming unit typically allows the drill-guide to be displaced along three axes. Precise-aiming unit 18 typically includes controls for precisely controlling the aforementioned rotations and/or translations of the drill-guide. Drill-guide mirrors (or non-mirrored surfaces) 36 and 38 are supported, respectively, by holders 84 and 88. Holders 84 and 88 are coupled, via bushings, to poles 82 and 86, respectively, the poles being coupled to drill-guide apparatus 10.

During a drill-guide positioning procedure, as described herein, mirrors 36 and 38 are fastened with respect to drill-guide apparatus 10, such that if the drill-guide is moved, the mirrors maintain their positions with respect to the drill-guide. The face of mirror 38 is rigidly fixed in the plane parallel to the drill-guide plane, i.e., the plane of at least two of the axes of drill-guide holes 44, 46 and 48. Mirror 36 is rigidly fixed such that it is perpendicular to the drill-guide plane. Typically, before the drill-guide positioning procedure, mirror 36 can be rotated by rotating holder 84 about pole 82. In some embodiments, the rotation angle of mirror 36 about pole 82 is determined by an angle-measuring device. In some embodiments, before the drill-guide positioning procedure, mirror 38 can be rotated about the by rotating holder 88 about pole 86. In some embodiments, there are cross-shaped hairlines 37 on mirror 38 and on mirror 36 (not shown). Alternatively, there are other markings on mirrors 36 and/or 38, and/or the mirrors are not circular. In some embodiments, instead of mirror 36, and/or instead of mirror 38, a non-mirrored surface is coupled to the drill-guide.

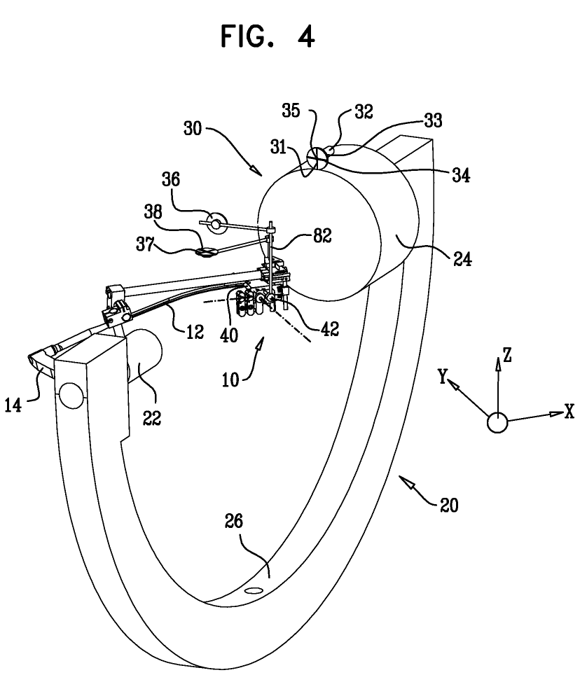

Reference is now made to FIG. 4, which is a schematic illustration of drill-guide apparatus 10 oriented with respect to an x-ray source 22 of x-ray machine 20, during a lateral x-ray, in accordance with an embodiment of the present invention. X-ray machine 20 typically includes x-ray source 22, an x-ray receiver 24, and a c-arm 26 coupling the x-ray source to the receiver. The drill-guide apparatus includes a coupling device that couples the drill-guide to the x-ray machine, to facilitate orientation of x-ray source 22 of the x-ray machine, with respect to the drill-guide. Typically, during a lateral x-ray, the coupling device couples the drill-guide to the x-ray machine such that the drill-guide plane is parallel to a central ray of an x-ray beam emitted by x-ray source 22. In some embodiments, the coupling device is an optical-coupling device 30 that optically couples the drill-guide to the x-ray source. For example, the optical-coupling device may include a light source 32 (e.g., a laser source, or an LED) physically coupled to x-ray receiver 24, an x-ray machine mirror 34 (or a non-mirrored surface) physically coupled to the x-ray receiver, and/or drill-guide mirrors 36 and 38 physically coupled to drill-guide apparatus 10. In some embodiments, the light source is physically coupled to the drill-guide (e.g., at mirror 36). Alternatively the coupling device is a non-optical-coupling device, for example, a mechanical coupling device, or an RF coupling device.

In some embodiments, laser source 32 is switched on, directing a light beam to mirror 36 though a hole 35 in mirror 34. The light beam reflected from mirror 36 appears in mirror 34 in the form of a light spot, which is observable by an operator. X-ray machine 20 is displaced until coincidence of the light spot with hole 35 in x-ray machine mirror 34 is attained. Aligning the light spot of the reflected beam with hole 35 of x-ray mirror 34 generally ensures that the mirrors 34 and 36 are parallel to each other. X-ray machine mirror 34 is typically coupled to the x-ray machine by an x-ray machine mirror housing 33, which couples the x-ray machine mirror to the x-ray machine such that the face of the x-ray machine mirror is contained in a plane that is perpendicular to the central ray of the x-ray beam. Since drill-guide mirror 36 is parallel to the x-ray machine mirror, and drill-guide mirror 36 is perpendicular to the drill-guide plane, this results in the drill-guide plane being parallel to the central ray of the x-ray beam. In some embodiments, instead of mirror 34, a non-mirrored surface is physically coupled to the x-ray machine. In some embodiments, using a non-mirrored surface instead of a mirror prevents the light beam being reflected multiple times, which may result in a user having difficulty aligning the incident light beam and the reflected light beam. In some embodiments, a light source is physically coupled to the drill-guide, instead of, or in addition to, being coupled to the x-ray machine. For some applications, instead of mirror 36, a non-mirrored surface is physically coupled to the drill-guide.

In some embodiments, x-ray machine 20 is displaced such that the laser beam reflects from the center of drill-guide mirror 36. This ensures that the centers of x-ray machine mirror 34 and drill-guide mirror 36 are aligned. In some embodiments, hairlines 31 of x-ray machine mirror 34 are aligned with hairlines (not shown) of drill-guide mirror 36. Typically, this ensures that a projection of drill-guide 40 will appear in a central portion of the lateral x-ray image generated by the x-ray machine. (Note that without this added step of alignment, it is possible for the two mirrors to be verified as parallel using the laser beam, but for the projection of drill-guide 40 to not be in the central portion of the lateral x-ray image, if, for example, c-arm 26 is not in the position shown in FIG. 4, but instead rotated 45 degrees.) Further typically, this ensures that when the drill guide is aligned with the nail hole, a central portion of the x-ray beam coincides with a point at which a line corresponding to the longitudinal axis of the drill guide coincides with the nail hole.

In some embodiments, the x-ray machine mirror and or the drill-guide mirror have other markings in order to facilitate a given rotational alignment of the mirrors. For some applications, the x-ray machine mirror, and/or the drill-guide mirror have non-circular shapes, for example, rectangular shapes, to facilitate rotational alignment of the mirrors. Alternatively other means are used for facilitating rotational alignment of the mirrors, as would be apparent to one skilled in the art. For example, two parallel light beams may be emitted from light-sources that are coupled to one of the mirrors and may be directed toward markings on the other mirror.

In some embodiments, before the drill-guide positioning procedure, x-ray machine 20 is rotated, such that the line extending between source 22 and receiver 24 is aligned in a desired direction (although this line will ultimately be displaced without rotation prior to acquisition of an x-ray image). The desired direction of the x-ray machine is typically such that a projection of the length of drill-guide 40 is shown in the lateral x-ray image (i.e., in the lateral x-ray image, a projection of the drill-guide is not shown looking down the length of the drill-guide, in which case the drill-guide would look like a circle). In some embodiments, the desired direction is a direction such that (a) a limb of the subject's body, in which the nail is not implanted does not appear in the x-ray image, without (b) the subject needing to adopt a position that is substantially uncomfortable. For example, for the subject shown in FIG. 2, the desired direction may be a direction, as shown, such that (a) the subject's left leg will not appear in the lateral x-ray image, without (b) the subject needing to adopt an uncomfortable position, for example, by needing to raise his left leg throughout the procedure.

It is noted that, with reference to the subject shown in FIG. 2, if a lateral x-ray is acquired along the longitudinal axis of the drill-guide (e.g., in accordance with prior art techniques), then, in order for the subject's left leg not to appear in the x-ray image, the c-arm of the x-ray machine is placed between the subject's legs at the level of the subject's right femur, and/or the subject's left leg must be raised. Both of these positions could cause substantial discomfort to the patient and/or complicate the procedure (by, for example, requiring the patient's body to be moved at one or more times during the x-ray acquisition procedure).

The x-ray machine having been rotated, by a given angle, mirror 36 is rotated around pole 82, by a corresponding angle. Subsequently the position of the x-ray machine is adjusted, using the optical-coupling device, so that mirror 34 is parallel to and aligned with mirror 36, as described hereinabove.

Subsequent to the orienting of x-ray machine 20 with respect to the drill-guide, a lateral x-ray image is acquired. As described hereinabove, using the optical coupling device ensures that (a) the central ray of the x-ray beam is parallel to the drill-guide plane, and (b) the drill-guide appears in the central portion of the x-ray image. Therefore, in accordance with the thought experiment described hereinabove, it may be determined from the lateral x-ray image whether the drill-guide and the distal end of the nail are aligned in a common plane by determining that, in the lateral x-ray image:
(a) a line corresponding to a longitudinal axis of the drill-guide and
(b) a line corresponding to a longitudinal axis of the distal end of the nail are substantially collinear.

Typically, it will be determined from the first lateral x-ray image that the drill-guide and the distal end of the nail are not aligned in a common plane. Therefore the position of the drill-guide 40 is corrected using the x-ray image, in order to align the drill-guide with nail-hole 54. In some embodiments, a drill or simulated drill 42 is placed in drill-guide 40 during the acquisition of lateral and/or frontal x-ray images.

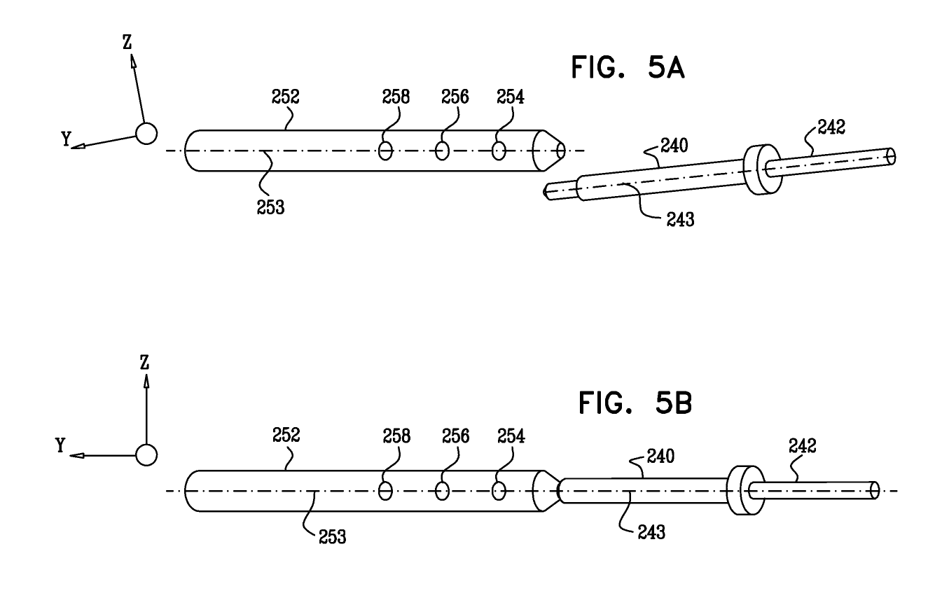

FIGS. 5A-B are schematic illustrations of the view from x-ray source 22, which would produce lateral x-ray images of projections 240 and 252 of drill-guide 40 and distal end 52 of nail 12, when the drill-guide is, respectively, misaligned and aligned with nail-hole 54 at the distal end of the nail, in accordance with an embodiment of the present invention. (In the actual x-ray images, less structural detail would be visible.) As seen in FIG. 5A, projection 240 of drill-guide 40 and projection 242 of drill 42 appear as elongated coaxial figures with parallel sides. A projection 252 of distal end 52 of nail 12 also appears as an elongated figure, including regions 254, 256, and 258 corresponding to nail-holes 54, 56, and 58. In the lateral x-ray photograph, a line 253 that corresponds to the longitudinal axis of distal end 52 of nail 12 intersects the axes of regions 254, 256 and 258 corresponding to nail-holes 54, 56 and 58, respectively. A line 243 that corresponds to the longitudinal axis of drill-guide 40 coincides with the axis of projection 240 of drill-guide 40. Typically, in the first lateral x-ray image, lines 243 and 253 are not substantially collinear, since the drill-guide is not aligned with nail-hole 54. Typically, the position of the drill-guide is adjusted based on the first lateral x-ray image.

In some embodiments, the angle between lines 243 and 253 is measured with a protractor or another angle-measuring device. The obtained angle is an input to a correction function, which is empirically or geometrically determined based on the physical characteristics of the apparatus. Drill-guide 40 is rotated through a correction angle, which is the output of the correction function, using precise aiming unit 18. The correction function typically has an input indicative of the fact that the x-ray was generated not along the y-axis, but instead at a given angle about the z-axis. In some embodiments, a table of data is provided for use with drill-guide apparatus 10, the table providing data regarding the appropriate manner in which to move the drill-guide, based on a given x-ray image.

In some embodiments, having rotated the drill-guide through the correction angle, the position of x-ray machine 20 is adjusted to correspond to the adjusted position of the drill-guide, using the optical-coupling device. Subsequently, a second lateral x-ray image is acquired. (Alternatively, having rotated the drill-guide through the correction angle, the new position of line 243 is drawn on the first x-ray image, to facilitate subsequent adjustments of the drill-guide.) The drill-guide having been rotated through the correction angle, line 243 in its new position either appears as parallel to line 253, or lines 243 and 253 coincide. If lines 243 and 253 do not coincide, the distance between the lines is measured, and drill-guide 40 is displaced along a graduated scale using precise aiming unit 18, in order that lines 243 and 253 should coincide.

In some embodiments, a computer is used to determine the angle through which the drill-guide should be rotated, and/or the distance through which the drill-guide should be translated based on the first lateral x-ray image. For example, an operator may enter data regarding the first x-ray image into the computer, and the computer calculates the appropriate angle and/or distance. Or, the first x-ray image may be provided as an input to the computer, the computer determining the manner in which the drill-guide should be moved, based on the first x-ray image. In an embodiment, the operator adjusts the position of the drill-guide through trial and error, based on the lateral x-ray image.

Typically, subsequent to the position of the drill-guide having been corrected, the position of the x-ray machine is adjusted accordingly, using the optical-coupling device. A further lateral x-ray image of drill-guide 40 and distal end 52 of nail 12 is acquired, in order to confirm that the drill-guide is aligned in the y-z plane with nail-hole 54 of the nail. The alignment in the y-z plane of the drill-guide with the hole is determined by determining that line 243 of projection 240 of the drill-guide is substantially collinear with line 253 of projection 252 of the distal end of the nail, as shown in FIG. 5B.

As described hereinabove, typically a series of more than one lateral x-ray is acquired, and drill-guide apparatus 10 and x-ray machine 20 are moved between successive lateral x-rays of the series. For some applications, before each of the lateral x-ray images is acquired, the alignment of a portion of optical coupling device 30 (e.g., light source 32) with the central ray of the x-ray beam is recalibrated. For example, the recalibration may be performed in order to account for movement of the light source with respect to c-arm 26 of the x-ray machine. Alternatively or additionally, the recalibration may be performed in order to account for shifting of the central ray of the x-ray beam with respect to the c-arm, due to the movement of the c-arm. Therefore, subsequent to the movement of the drill-guide and the x-ray machine, the orientation of the central ray of the x-ray beam is determined, in accordance with techniques that are known in the art. In addition, the orientation of a light beam that is generated by the light source is determined. In response to determining that the light beam that is generated by the light source is not aligned with the central ray of the x-ray beam, the disposition of the light source with respect to the x-ray source is adjusted accordingly. For applications in which a different optical coupling device is used, the optical coupling device is typically recalibrated with respect to the x-ray source before each of the lateral x-ray images is acquired, as described hereinabove, mutatis mutandis

Reference is now made to FIG. 6, which is a schematic illustration of drill-guide apparatus 10 oriented with respect to x-ray source 22 of x-ray machine 20 during a frontal x-ray, in accordance with an embodiment of the present invention. Optical-coupling device 30, or another coupling device, is used to orient the x-ray source with respect to the drill-guide, for the frontal x-ray image acquisition. In embodiments in which optical-coupling device 30 is used, the x-ray machine is typically positioned such that (a) the incident light beam generated by source 32 coincides with the beam that is reflected from mirror 38, and/or (b) crosshairs, or other markings on mirrors 38 and 34 coincide. The coupling of the drill-guide to the x-ray machine is generally similar to that described with respect to the coupling of the drill-guide to the x-ray machine for the purposes of the lateral x-ray, as described hereinabove. In some embodiments, a non-mirrored surface is used instead of either mirror 38 or mirror 34. In some embodiments, a light source is physically coupled to the drill-guide, instead of being physically coupled to the x-ray machine. Typically, during a frontal x-ray, the coupling device is used to ensure that (a) a projection drill-guide will appear in a central portion of the frontal x-ray image, and (b) the drill-guide plane is perpendicular to a central ray of the x-ray beam. Typically, the result of (a) increases the accuracy of the x-ray image and reduces distortion due to imaged elements not being in the center of the x-ray image. Once the x-ray source has been oriented with respect to the drill-guide, a frontal x-ray image is acquired.

It is noted that in some embodiments, frontal x-ray images of the drill-guide and the nail are acquired in accordance with prior art techniques, and lateral x-ray images are acquired in accordance with the techniques described herein. Furthermore, in some embodiments, alignment, in the x-y plane, of drill-guide 40 with nail-hole 54 of distal end 52 of nail 12 is performed in accordance with prior art techniques. For some applications, lateral x-ray images of the drill-guide and the nail are acquired in accordance with prior art techniques, and frontal x-ray images are acquired in accordance with the techniques described herein. In some embodiments, alignment, in the y-z plane, of drill-guide 40 with nail-hole 54 of distal end 52 of nail 12, is performed in accordance with prior art techniques.

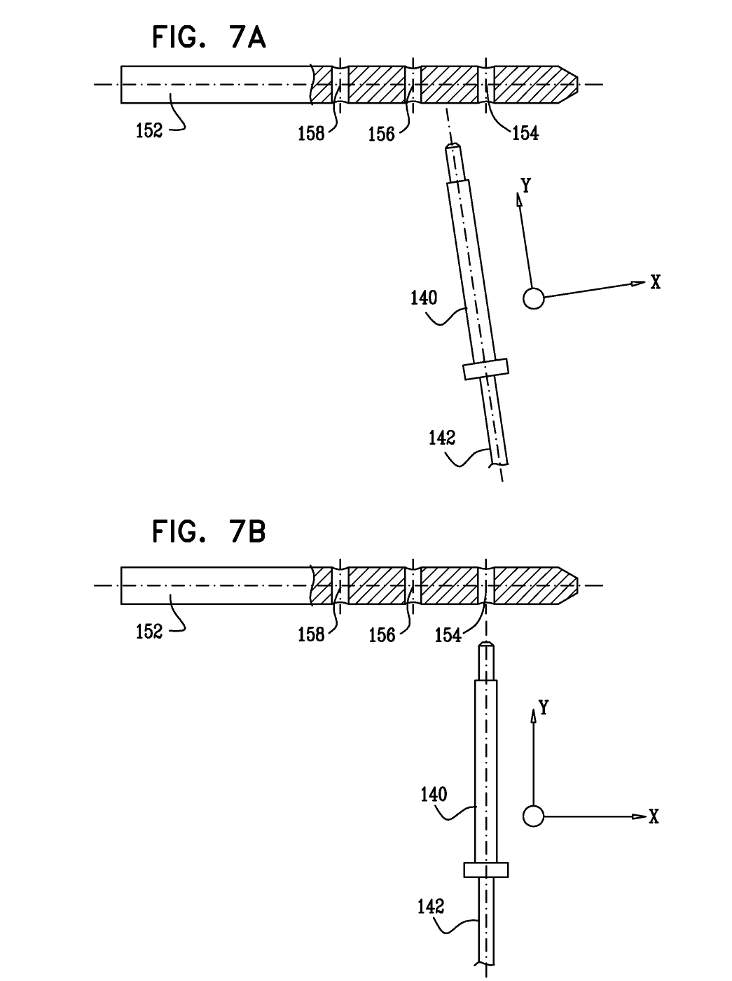

Reference is now made to FIGS. 7A-B, which are schematic illustrations of the view from x-ray source 22, which would produce frontal x-ray images of projections 140 and 152 of drill-guide 40 and distal end 52 of nail 12, when the drill-guide is respectively misaligned and aligned with a hole at the distal end of the nail, in accordance with an embodiment of the present invention. (In the actual x-ray images, less structural detail would be visible.) Projection 152 of the distal end of the nail includes axes 154, 156, and 158 corresponding to longitudinal axes of nail-holes 54, 56, and 58. Typically, in the first frontal x-ray image that is acquired, projection 140 of the drill-guide and axis 154, corresponding to the longitudinal axis of nail-hole 54, are not substantially collinear, as shown in FIG. 7A.

In response to the first x-ray image, the position of the drill-guide is adjusted, by translating and/or rotating the drill-guide. The position of the drill-guide having been adjusted, the position of x-ray machine 20 is adjusted to correspond to the adjusted position of the drill-guide, using the coupling device. The correction of the position of the drill-guide in response to the frontal x-ray image is generally similar, mutatis mutandis, to the correction of the position described hereinabove, in response to the lateral x-ray image. The position of the drill-guide is corrected such that projection 140 appears substantially collinear to the axis 154, corresponding to the longitudinal axis of nail-hole 54, in a frontal x-ray image, as shown in FIG. 7B.

As described hereinabove, typically, during the frontal x-ray imaging procedure, the position of the x-ray machine is adjusted in correspondence to adjustments to the position of the drill-guide. In some embodiments, the result of this is that when an x-ray image is observed in which projection 140 appears substantially collinear to the projection 154, that image has been acquired using an x-ray source positioned such that the central ray of the x-ray beam emitted by the source is perpendicular to the longitudinal axis of nail-hole 54.

As described hereinabove, typically a series of more than one frontal x-ray is acquired, and drill-guide apparatus 10 and x-ray machine 20 are moved between successive frontal x-rays. For some applications, before each of the frontal x-ray images is acquired, the alignment of a portion of optical coupling device 30 (e.g., light source 32) with the central ray of the x-ray beam is recalibrated, in a generally similar manner to that described hereinabove with reference to the lateral x-ray images.

In some embodiments, to increase the precision of the positioning of the drill-guide, a drill or simulated drill 42 is placed into drill-guide 40, and a projection 142 of the drill or simulated drill appears in the frontal and/or lateral x-ray images.

Typically, once it has been confirmed, using frontal and lateral x-ray images, that drill-guide 40 is aligned with nail-hole 54, drill 42, or another bone-penetration device, is inserted into drill-guide 40, and bone drilling is performed. Subsequently, drill 42 and drill-guide 40 are withdrawn from drill-guide hole 44, which faces nail-hole 54, and a screw, or a different nail fastener, is screwed through drill-guide hole 44 into nail-hole 54 using a screw guide (screw and screw guide not shown). Drill-guide 40 is then placed into the drill-guide hole 46, which faces nail-hole 56 in the distal end 52 of the nail, and operations connected with bone drilling and screwing of the screw are repeated. Finally, the drill-guide is transferred into drill-guide hole 48, which is opposite nail-hole 58 in distal end 52 of the nail, and all operations connected with bone drilling and screwing of the screw are again repeated.

It is noted that in some embodiments, alignment of drill-guide 40 with nail-hole 54, in the x-y plane, using frontal x-ray images, is performed before alignment of the drill-guide with the nail-hole, in the y-z plane, using lateral x-ray images.

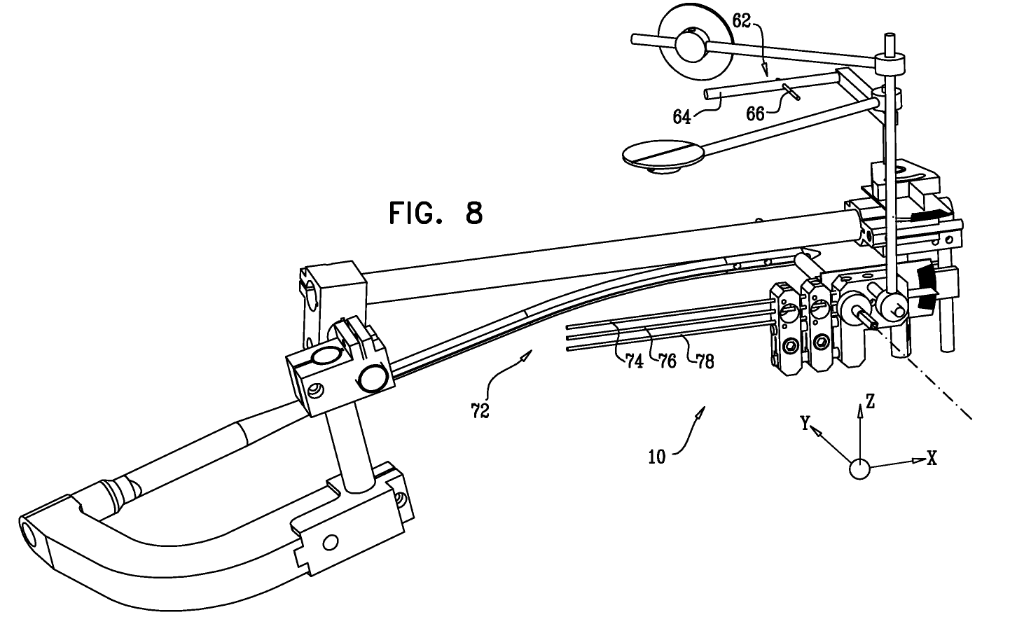

Reference is now made to FIG. 8, which is a schematic illustration of drill-guide apparatus 10, including radiopaque indicating elements 64, 66, 74, 76, and 78, in accordance with an embodiment of the present invention. In some embodiments, the positioning of drill-guide 40 with respect to nail-hole 54 is facilitated by one or more of the radiopaque indicating elements. In some embodiments, the radiopaque indicating elements are used to facilitate positioning of the drill-guide, when the projection of the drill-guide does not appear clearly in frontal and/or lateral x-ray images. An orienting device 62, which includes indicating elements 64 and 66, is typically used to facilitate positioning of drill-guide 40 using frontal x-ray images. An orienting device 72, which includes indicating elements 74, 76, and 78, is typically used to facilitate positioning of drill-guide 40 using lateral x-ray images.

In some embodiments, indicating elements 64 and 66 are two perpendicular rods coupled to apparatus 10, such that rod 64 is perpendicular to the axis of drill-guide 40, and rod 66 is parallel to the axis of drill-guide 40. Orienting device 62 is preliminarily set in such a way relatively to drill-guide 40 that the axis of the projection of rod 66 in a frontal x-ray image coincides with the axis of the projection of drill-guide 40.

In some embodiments, indicating elements 74, 76, and 78 include three parallel rods located at equal distances from each other and coupled to apparatus 10. Rod 76 is preliminarily set in such a way relative to drill-guide 40 that, when the drill-guide is ultimately correctly aligned with nail-hole 54, the axis of the projection of rod 76 in a lateral x-ray image coincides with the axis of the projection of distal end 52 of nail 12.

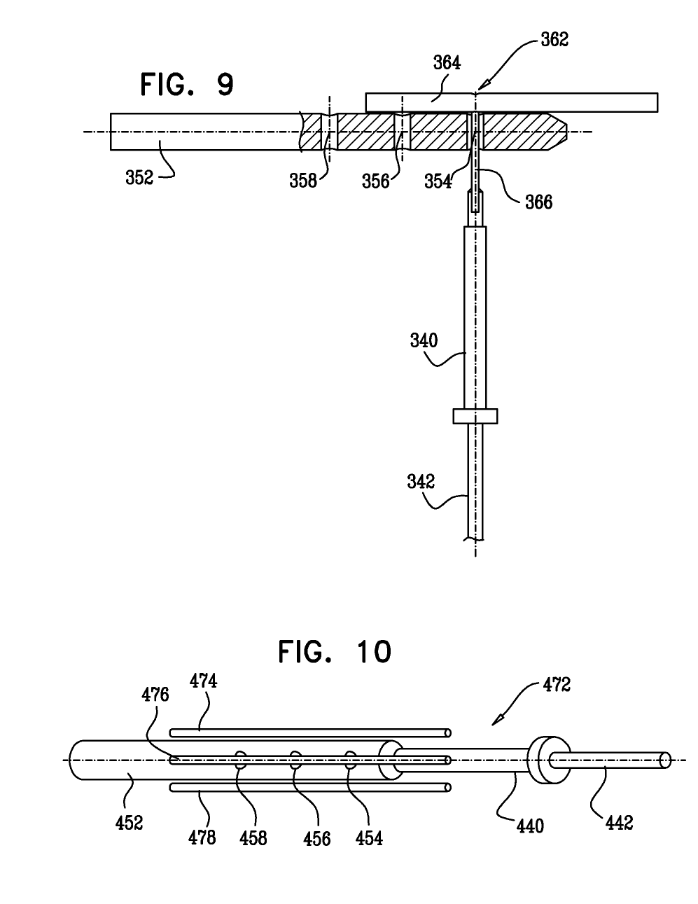

Reference is now made to FIG. 9, which is a schematic illustration of the view from x-ray source 22, which would produce a frontal x-ray image of projections 340, 352, 364, and 366 of, respectively, drill-guide 40, distal end 52 of the nail, and the radiopaque indicating elements 64 and 66, in accordance with an embodiment of the present invention. (In the actual x-ray image, less structural detail would be visible.)

The drill-guide position with respect to nail-hole 54 may be located in the frontal x-ray image by locating projection 366 of rod 66 in the frontal x-ray image. At a correct alignment of drill-guide 40, projection 366 of rod 66 is located centro-symmetrically along longitudinal axis 354 that corresponds to the longitudinal axis of nail-hole 54. When the drill-guide is correctly aligned, projection 364 of rod 64 appears parallel and adjacent to projection 352 of distal end 52 of the nail, as shown.

Reference is now made to FIG. 10, which is a schematic illustration of the view from x-ray source 22, which would produce a lateral x-ray image of projections 440, 452, 474, 476 and 478 of, respectively, drill-guide 40, distal end 52 of the nail, and the radiopaque indicating elements 74, 76, and 78, in accordance with an embodiment of the present invention. (In the actual x-ray image, less structural detail would be visible.)

The position of drill-guide 40 is located by locating the positions of projections 474, 476 and 478 of rods 74, 76 and 78, respectively, in the lateral x-ray image. At a correct alignment of drill-guide 40 with respect to nail-hole 54 of the nail, projections 474, 476 and 478 of rods 74, 76 and 78, respectively, are parallel to projection 452 of distal end 52 of nail 12 (as shown). The axis of projection 476 of rod 76 coincides with the axis of projection 452 of distal end 52 of nail 12.

In some embodiments, lateral x-ray images are acquired by aligning the x-ray source with the longitudinal axis of the drill-guide. In such embodiments, drill-guide apparatus 10 includes radiopaque indicating elements, for example, elements 74, 76, and 78. Drill-guide 40, nail-hole 54, and the radiopaque indicating elements appear in the x-ray image as circles, and the radiopaque elements are used to facilitate alignment of the drill-guide with nail hole 54.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for aligning a bone-penetration device with a nail-hole at a distal end of an implantable nail, the method comprising:
generating at least one lateral x-ray image of a drill-guide and the distal end of the nail by:
orienting an x-ray source of an x-ray machine with respect to the drill-guide, using a coupling device that couples the x-ray machine to the drill-guide; and
generating at least one lateral x-ray image of the drill-guide and the distal end of the nail, using the x-ray source, while the x-ray source is oriented with respect to the drill-guide;
determining that the drill-guide is aligned with the nail-hole by determining that, in the lateral x-ray image:
(a) a line corresponding to a longitudinal axis of the drill-guide and
(b) a line corresponding to a longitudinal axis of the distal end of the nail are substantially collinear; and
aligning the bone-penetration device using the drill-guide, in response to determining that the drill-guide is aligned with the nail-hole.

2. The method according to claim 1, wherein determining that the drill-guide is aligned with the nail-hole further comprises determining that, in the x-ray image, a projection of a radiopaque indicating element that is coupled to the drill-guide appears in a predefined geometrical relationship with respect to a projection of the distal end of the nail.

3. The method according to claim 1, wherein orienting the x-ray source of the x-ray machine with respect to the drill-guide comprises orienting the x-ray source such that, when the drill guide is aligned with the nail hole, a central portion of the x-ray beam coincides with a point at which the line corresponding to the longitudinal axis of the drill guide coincides with the nail hole.

4. The method according to claim 1, wherein orienting the x-ray source with respect to the drill-guide comprises optically coupling the drill-guide to the x-ray machine using an optical-coupling device that optically couples the x-ray machine to the drill-guide.

5. The method according to claim 4, wherein using the optical-coupling device comprises detecting that an incident light beam emitted by a light source of the optical-coupling device coincides with a reflection of the incident light beam that is reflected from a mirror of the optical-coupling device.

6. The method according to claim 4, wherein using the optical-coupling device comprises determining that a mirror of the optical-coupling device that is physically coupled to the drill-guide is aligned with a mirror of the optical-coupling device that is physically coupled to the x-ray machine.

7. The method according to claim 4, wherein using the optical-coupling device comprises determining that a mirror of the optical-coupling device that is physically coupled to the drill-guide is aligned with a non-mirrored surface of the optical-coupling device that is physically coupled to the x-ray machine.

8. The method according to claim 4, wherein using the optical-coupling device comprises determining that a non-mirrored surface of the optical-coupling device that is physically coupled to the drill-guide is aligned with a mirror of the optical-coupling device that is physically coupled to the x-ray machine.

9. The method according to claim 1, wherein in generating the at least one lateral x-ray image and determining that the drill-guide is aligned with the nail-hole, the method comprises:
generating a first lateral x-ray image, while the drill-guide is in a first drill-guide position, and while the x-ray source is in a first x-ray source position;
using the first lateral x-ray image, determining if the drill-guide is not aligned with the nail-hole;
in response to determining that the drill-guide is not aligned with the nail-hole, moving the drill-guide from the first drill-guide position to a second drill-guide position, in order to align the drill-guide with the nail-hole;
using the coupling device, moving the x-ray source from the first x-ray source position to a second x-ray source position, such that the x-ray source is oriented with respect to the drill-guide, while the drill-guide is in the second drill-guide position;
while the drill-guide is in the second drill-guide position, and the x-ray source is in the second x-ray source position, generating a second lateral x-ray image of the drill-guide and the distal end of the nail, using the x-ray source; and
determining that the drill-guide is aligned with the nail-hole using the second x-ray image.

10. The method according to claim 9, wherein moving the drill-guide from the first drill-guide position to the second drill-guide position comprises, in response to the first x-ray image, moving the drill-guide in accordance with drill-guide motion data disposed in a table of drill-guide motions.

11. The method according to claim 9, wherein moving the drill-guide from the first drill-guide position to the second drill-guide position comprises, in response to the first x-ray image, determining a manner in which to move the drill-guide using a computer.

12. The method according to claim 11, wherein determining the manner in which to move the drill-guide using the computer comprises providing the first x-ray image as input to the computer.

13. The method according to claim 1,
wherein the drill-guide includes a drill-guide that is insertable through at least two drill-guide holes of at least one drill-guide holder, longitudinal axes of the drill-guide holes being parallel to each other and defining a drill-guide plane, and
wherein orienting the drill-guide with respect to the x-ray source comprises placing the drill-guide such that a central ray of an x-ray beam of the x-ray source is in an orientation selected from the group consisting of: parallel to the drill-guide plane, and in the drill-guide plane.

14. The method according to claim 13, wherein the nail includes a nail that defines at least first and second nail-holes at the distal end thereof, and wherein determining that the drill-guide is aligned with the nail-hole comprises determining that a second drill-guide hole of the at least two drill-guide holes is aligned with the second nail-hole, by determining that a first drill-guide hole of the at least two drill-guide holes is aligned with the first nail-hole.

15. The method according to claim 1, further comprising:
orienting an x-ray source of an x-ray machine with respect to the drill-guide, using a coupling device that couples the x-ray machine to the drill-guide; and
generating at least one frontal x-ray image of the drill-guide and the distal end of the nail, using the x-ray source, while the x-ray source is oriented with respect to the drill-guide,
wherein determining that the drill-guide is aligned with the nail-hole further comprises determining that, in the frontal x-ray image, the drill-guide and the nail-hole are substantially collinear.

16. The method according to claim 15,
wherein the drill-guide includes a drill-guide that is insertable through at least two drill-guide holes of at least one drill-guide holder, longitudinal axes of the drill-guide holes being parallel to each other and defining a drill-guide plane, and
wherein orienting the drill-guide with respect to the x-ray source comprises orienting the drill-guides such that the drill-guide plane is a plane that is perpendicular to a central ray of an x-ray beam of the x-ray source.

17. Apparatus for aiming a bone-penetration device and for use with (a) an x-ray machine including an x-ray source and an x-ray receiver, and (b) a nail that is implanted in a bone of a subject, the nail defining at least one nail-hole at a distal end thereof, the apparatus comprising:
a drill-guide configured to align the bone-penetration device with the nail-hole; and
an optical coupling device that optically couples the drill-guide to the x-ray machine and is configured to facilitate orientation of the drill-guide with respect to the x-ray source, during acquisition of a lateral x-ray image, such that it may be determined that the drill-guide is aligned with the nail-hole by determining that, in the lateral x-ray image:
(a) a line corresponding to a longitudinal axis of the drill-guide and
(b) a line corresponding to a longitudinal axis of the distal end of the nail are substantially collinear.

18. The apparatus according to claim 17, further comprising at least one radiopaque indicating element coupled to the drill-guide and configured to facilitate verification of alignment of the drill-guide with the nail-hole by a projection of the indicating element appearing in an x-ray image of the nail in a predefined geometrical relationship with respect to the nail when the drill-guide is properly aligned with the nail-hole.

19. The apparatus according to claim 17, wherein the coupling device comprises an optical-coupling device comprising a first portion physically coupled to the drill-guide and a second portion physically couplable to the x-ray machine, and configured to couple the drill-guide to the x-ray machine by optically coupling the x-ray machine to the drill-guide.

20. The apparatus according to claim 19, wherein the first portion of the optical-coupling device comprises a drill-guide mirror that is physically coupled to the drill-guide.

21. The apparatus according to claim 20, wherein the second portion of the optical-coupling device comprises a light source, and wherein the optical-coupling device is configured to facilitate the orientation of the drill-guide with respect to the x-ray source, by an incident light beam that is emitted from the light source coinciding with a reflected beam of the incident beam that is reflected from the mirror.

22. The apparatus according to claim 20, wherein the second portion of the optical-coupling device comprises a non-mirrored surface that is physically couplable to the x-ray machine.

23. The apparatus according to claim 20, wherein the drill-guide mirror comprises:
a first drill-guide mirror that is coupled to the drill-guide and configured to facilitate orientation of the x-ray source with respect to the drill-guide, during acquisition of a frontal x-ray that is taken by the x-ray machine, and
a second drill-guide mirror that is coupled to the drill-guide and configured to facilitate the orientation of the x-ray source with respect to the drill-guide, during acquisition the lateral x-ray that is taken by the x-ray machine.

24. The apparatus according to claim 20, wherein the second portion of the optical-coupling device comprises an x-ray machine mirror.

25. The apparatus according to claim 24, wherein the drill-guide mirror and the x-ray machine mirror have markings on respective faces thereof, the markings facilitating rotational alignment of the drill-guide mirror and the x-ray machine mirror.

26. The apparatus according to claim 19, wherein the second portion of the optical-coupling device comprises an x-ray machine mirror that is physically couplable to the x-ray machine.

27. The apparatus according to claim 26, wherein the first portion of the optical-coupling device comprises a non-mirrored surface that is physically coupled to the drill-guide.

28. The apparatus according to claim 26, further comprising an x-ray machine mirror housing, operative to physically couple the x-ray machine mirror to the x-ray machine such that a face of the x-ray machine mirror is contained in a plane that is perpendicular to the central ray of the x-ray beam.

29. The apparatus according to claim 26, wherein the first portion of the optical-coupling device comprises at least one light source that is physically coupled to the drill-guide, and wherein the optical-coupling device is configured to facilitate the orientation of the x-ray source with respect to the drill-guide, by an incident light beam that is emitted from the light source coinciding with a reflected beam of the incident beam that is reflected from the mirror.

30. The apparatus according to claim 17,
further comprising at least one drill-guide holder shaped to define at least first and second drill-guide holes, the drill-guide being configured to be inserted through the drill-guide holes, longitudinal axes of the first and second drill-guide holes being parallel to each other and defining a drill-guide plane,
wherein the coupling device is configured to facilitate orientation of the x-ray source with respect to the drill-guide, during acquisition of the lateral x-ray image, such that a central ray of an x-ray beam of the x-ray source is in an orientation selected from the group consisting of: parallel to the drill-guide plane, and in the drill-guide plane.

31. The apparatus according to claim 30, wherein the nail includes a nail that defines at least first and second nail-holes at the distal end thereof, and wherein a distance between the first drill-guide hole and the second drill-guide hole is the same as a distance between the first nail-hole and the second nail-hole.

32. The apparatus according to claim 17, wherein the coupling device is further configured to facilitate orientation of the x-ray source with respect to the drill-guide, during acquisition of a frontal x-ray image, such that it may be determined that the drill-guide is aligned with the nail-hole by determining that, in the frontal x-ray image, the drill-guide and the nail-hole are substantially collinear.

33. The apparatus according to claim 32,
further comprising at least one drill-guide holder shaped to define at least first and second drill-guide holes, the drill-guide being configured to be inserted through the drill-guide holes, longitudinal axes of the first and second drill-guide holes being parallel to each other and defining a drill-guide plane,
wherein the coupling device is configured to facilitate orientation of the x-ray source with respect to the drill-guide, during acquisition of the frontal x-ray image, such that the drill-guide plane is a plane that is perpendicular to a central ray of an x-ray beam of the x-ray source.

34. A method for aligning a bone-penetration device with a nail-hole at a distal end of an implantable nail, the method comprising:
orienting an x-ray source of an x-ray machine with respect to a drill-guide, using a optical coupling device that optically couples the x-ray machine to the drill-guide;
generating a first frontal x-ray image of the drill-guide and the distal end of the nail, using the x-ray source, while the x-ray source is oriented with respect to the drill-guide, while the drill-guide is in a first drill-guide position, and while the x-ray source is in a first x-ray source position;
using the first frontal x-ray image, determining if the drill-guide is not aligned with the nail-hole;
in response to determining that the drill-guide is not aligned with the nail-hole, moving the drill-guide from the first drill-guide position to a second drill-guide position, in order to align the drill-guide with the nail-hole;
using the coupling device, moving the x-ray source from the first x-ray source position to a second x-ray source position, such that the x-ray source is oriented with respect to the drill-guide, while the drill-guide is in the second drill-guide position;
while the drill-guide is in the second drill-guide position, and the x-ray source is in the second x-ray source position, generating a second frontal x-ray image of the drill-guide and the distal end of the nail, using the x-ray source;
determining that the drill-guide is aligned with the nail-hole by determining that, in the second frontal x-ray image, the drill-guide and the nail-hole are substantially collinear; and
aligning the bone-penetration device using the drill-guide, in response to determining that the drill-guide is aligned with the nail-hole.

35. The apparatus according to claim 34, wherein the x-ray machine is not physically coupled to the drill-guide.
